(19) United States
(12) Patent Application Publication
(10) Pub. No.: US 2005/0265955 A1
(43) Pub. Date: Dec. 1, 2005

(54) Sustained release preparations

(75) Inventors: Siva N. Raman, St. Louis, MO (US); John P. Cunningham, St. Louis, MO (US); John F. Lang, Florence, SC (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Mallinckrodt Inc., St. Louis, MO (US)

(21) Appl. No.: 10/857,533

(22) Filed: May 28, 2004

(51) Int. Cl. A61K 31/785

(52) U.S. Cl. 424/78.12

(57) ABSTRACT

Disclosed are sustained release drug particles suitable for forming sustained release oral pharmaceutical compositions. The sustained release drug particles comprise a drug-ion exchange resin complex and a water-permeable, diffusion barrier surrounding at least a portion of the drug-ion exchange resin complex. The diffusion barrier comprises a film-forming polymer and is free or contains no substantial traces of organic solvent. Also disclosed are oral pharmaceutical compositions, for example, oral suspensions, comprising the sustained release drug particles, a method for the controlled administration of a drug to a patient, and a method for manufacturing the sustained release drug particles. The method of manufacturing involves the use of an aqueous coating composition comprising a water-permeable film-forming polymer such as ethylcellulose.

### FIELD OF THE INVENTION

This invention pertains to sustained release drug preparations, such as liquid suspensions, wherein a drug is complexed with an ion exchange resin particle and the drug-ion exchange resin complex is coated with a water-permeable film-forming polymer that serves as a diffusion barrier.

### BACKGROUND OF THE INVENTION

The treatment, control, and amelioration of disorders and/or the control of symptoms are basic goals of drug therapy. One aspect of such therapy is the sustained administration of an effective dose of drug for an extended period of time. In many cases, the longer the period of time, the more substantial the benefit. Sustained release is advantageous since patient's side effects arising out of administering an immediate release therapy are substantially reduced. Sustained or prolonged-release dosage forms of various drugs are known. In one example, the drug is complexed with an ion exchange resin forming a drug-ion exchange resin complex particle. After administration, the drug is slowly released from the complex over time thereby providing a continuous delivery of drug to the patient. In certain examples, the drug-ion exchange resin complex particle is coated with a diffusion barrier made of a water-permeable film. See, for example, U.S. Pat. Nos. 4,221,778 and 4,996,047. Such coated particles, however, have one or more drawbacks, for example, they may involve difficult or expensive to manufacture, e.g., requiring multiple steps and a polymer coating step wherein the polymer must be dissolved and applied from a non-aqueous (organic) solvent, a significant quantity of which, undesirably, remains in the final product as a residual solvent. Alternatively, high loadings of the drug in the ion exchange resin are required to maintain the integrity of the coating and to provide satisfactory performance.

The foregoing shows that there exists a need for a sustained release drug composition wherein the coated drug-ion exchange resin complex particle is free or substantially free of non-aqueous or organic solvents. There further exists a need for sustained release drug compositions wherein the coated drug-ion exchange resin complex particle can have any desired amount of drug loading and still provide excellent performance. There also exists a need for a manufacturing process that is economical and that does not employ substantial amounts of organic solvents.

The invention provides such a sustained release composition and a manufacturing process. The advantages of the invention, as well as additional inventive features, will be apparent from the description of the invention provided herein.

### BRIEF SUMMARY OF THE INVENTION

The foregoing needs have been fulfilled to a great extent by the present invention. Accordingly, the present invention provides sustained release drug particles suitable for forming sustained release pharmaceutical compositions. The sustained release drug particles comprise a drug-ion exchange resin complex and a water-permeable diffusion barrier surrounding at least a portion of the drug-ion exchange resin complex. The diffusion barrier comprises a film-forming polymer and is free or substantially free of traces of organic solvents, particularly harmful organic solvents. The present invention further provides oral pharmaceutical compositions comprising the sustained release drug particles. The present invention also provides a method for manufacturing the sustained release drug particles. The method involves the use of an aqueous coating composition comprising a water-permeable film-forming polymer. The present invention further provides a method for the controlled administration of a drug to a patient.

### DETAILED DESCRIPTION OF THE INVENTION

The present invention provides delayed release drug formulations comprising drug-ion exchange resin complex particles bearing a diffusion barrier. It has been surprisingly discovered that sustained release drug particles containing a pharmaceutically active drug can be manufactured using an aqueous carrier for the materials of the coating rather than non-aqueous carriers. The present invention has one or more advantages. For example, the particle manufacture is relatively simple or less expensive than processes involving organic solvents, and requires no substantial amounts of non-aqueous or organic solvent during manufacture or processing. The process produces cleaner, safer product. The present invention also advantageously provides sustained release drug particles having a drug loading that spans a wide range.

The present invention further provides a sustained release drug particle suitable for forming a sustained release oral pharmaceutical composition comprising a drug-ion exchange resin complex, and a water-permeable diffusion barrier surrounding at least a portion of the drug-ion exchange resin complex; wherein the diffusion barrier comprises a film-forming polymer and is free or substantially free of traces of organic solvents. In an embodiment, the diffusion barrier completely surrounds the drug-ion exchange resin complex. The drug-ion exchange resin complex optionally includes a solvating agent.

The present invention provides, in an embodiment, an oral pharmaceutical composition comprising ion exchange resin having particle sizes from about 30 microns to about 500 microns; at least one pharmaceutically active drug releasably bound to the particles to form a drug-ion exchange resin complex, wherein the drug-ion exchange resin complex is coated with an aqueous based diffusion barrier which comprises from about 1% to about 60% by weight of the resin particles, of a water-permeable film-forming polymer. In an embodiment, the water-permeable film-forming polymer contains no substantial traces of an organic solvent. The drug is released from the complex under the conditions of the gastrointestinal fluids, e.g., pH, ionic strength, and the type of ions present therein.

Any suitable ion exchange resin can be used. Ion exchange resins suitable for use in these preparations are water-insoluble and comprise a pharmacologically inert organic and/or inorganic matrix containing covalently bound functional groups that are ionic or capable of being ionized under the appropriate conditions, e.g., pH. The organic matrix may be synthetic (e.g., polymers or copolymers of acrylic acid, methacrylic acid, sulfonated styrene, and sulfonated divinylbenzene) or partially synthetic (e.g., modified cellulose and dextrans). An example of the inorganic matrix is silica gel modified by the addition of ionic groups. Covalently bound ionic groups may be strongly acidic (e.g., sulfonic acid or phosphonic acid), weakly acidic (e.g., carboxylic acid), strongly basic (e.g., primary amine), weakly basic (e.g., quaternary ammonium), or a combination of acidic and basic groups. In general, the types of ion exchange resins suitable for use in ion-exchange chromatography and for such applications as deionization of water are suitable for use in the controlled release of drug preparations. Ion exchange resins having any suitable ion exchange capacity can be employed, e.g., ion exchange resins having ion exchange capacities below about 6 milliequivalents (meq)/gram, preferably below about 5.5 meq/gram, and more preferably from about 3 to about 5 meq/gram. The ion exchange resins are typically crosslinked, for example, to a degree of crosslinking about 4% or more, e.g., from about 4% to about 20%, preferably from about 4% to about 16%, and more preferably from about 6% to about 12%.

The ion exchange resin particles can have any suitable size, e.g., a size less than about 1000 microns. Typically, the size of the ion exchange resin particles is from about 30 microns to about 500 microns, preferably from about 40 microns to about 200 microns, and more preferably from about 50 microns to about 150 microns, for liquid and solid dosage forms, although particles having sizes up to about 1,000 microns can be used for solid dosage forms, e.g., tablets and capsules.

In accordance with embodiments of the present invention, both regularly shaped and irregularly shaped ion exchange resin particles may be used. Regularly shaped particles include those particles that substantially conform to well-defined geometric shapes such as spherical, elliptical, and cylindrical, cubic, rhombohedral, and the like. An example of a spherical ion exchange resin is DOWEX™ 50WX8H (Dow Chemical Co.), which is a strong acid cation exchange resin having 8% degree of crosslinking, with sulfonic acid functional groups (sulfonated polystyrene-divinylbenzene), an ion exchange capacity of 4.2 meq/gram, and a particle size of 45 microns to 150 microns. Irregularly shaped particles are all particles not considered to be regularly shaped, such as particles with amorphous shapes and particles with increased surface areas due to surface channels or distortions. An example of an irregularly shaped ion exchange resin particle is AMBERLITE™ IRP-69 (Rohm and Haas Co.), which is also a sulfonated polymer (sulfonated polystyrene-divinylbenzene) but in the sodium salt form. AMBERLITE IRP-69 resin particles have a size of 37 microns to 149 microns, a degree of crosslinking of 8%, and an ion exchange capacity of 4.2 meq/gram. Preferably, the ion AMBERLITE IRP-69 exchange resin is sieved to obtain a particle size of from 75 microns to 149 microns.

In accordance with the present invention, any suitable pharmaceutically or pharmacologically active drug can be prepared as a sustained release formulation. Examples of such drugs include drugs for the treatment of respiratory tract disorders such as, for example, antitussive expectorants such as dihydrocodeine phosphate, codeine phosphate, noscapine hydrochloride, phenylpropanolamine hydrochloride, potassium guaiacolsulfonate, cloperastine fendizoate, dextromethorphan hydrobromide and chloperastine hydrochloride; bronchodilators such as dl-methylephedrine hydrochloride and dl-methylephedrine saccharinate; and antihistamines such as fexofenadine HCl or dl-chlorpheniramine maleate. Other drugs useful for the invention include drugs for the treatment of digestive tract disorders such as, for example, digestive tract antispasmodics including scopolamine hydrobromide, metixene hydrochloride and dicyclomine hydrochloride, drugs for the treatment of central nervous system disorders such as, for example, antipsychotic drugs including phenothiazine derivatives (e.g., chlorpromazine hydrochloride) and phenothiazine-like compounds (e.g., chlorprothixene hydrochloride), antianxiety drugs such as benzodiazepine derivatives (e.g., chlordiazepoxide hydrochloride, diazepam), antidepressants such as imipramine compounds (e.g., imipramine hydrochloride), antipyretic analgesics such as sodium salicylate, and hypnotics such as phenobarbital sodium; opioid analgesic drugs such as alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethotheptazine, ethylmethylthiambutene, ethylmorphine, etonitazene fentanyl, heroin, hydrocodone, hydromorphone, hydroxypethidine, isomethadone, ketobemidone, levallorphan, levorphanollevophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavretum, pentazocine, phenadoxone, phenomorphan, phenazocine, phenoperidine, piminodine, piritramide, propheptazine, promedol, properidine, propiram, propoxyphene, sufentanil, tramadol, tilidine, pharmaceutically acceptable salts thereof, mixtures of any of the foregoing, mixed μ-agonists/antagonists, μ-antagonist combinations, and the like; and drugs for the treatment of respiratory system disorders such as, for example, coronary dilators including etafenone hydrochloride, antiarrhythmics such as procainamide hydrochloride, calcium antagonists such as verapamil hydrochloride, hypotensive drugs such as hydrazine hydrochloride, propranolol hydrochloride and clonidine hydrochloride, and peripheral vasodilators/vasoconstrictors such as tolazoline hydrochloride. Antibiotics that may be useful include macrolides such as oleandomycin phosphate, tetracyclines such as tetracycline hydrochloride, streptomycins such as fradiomycin sulfate, and penicillin drugs such as dicloxacillin sodium, pivmecillinam hydrochloride and carbenicillinindanyl sodium. Chemotherapeutic drugs may also be used including sulfa drugs such as sulfisomidine sodium; antituberculosis drugs such as kanamycin sulfate, and antiprotozoan drugs such as amodiaquine hydrochloride. An excellent sustained releasing effect is obtained in basic drugs for the respiratory tract such as dihydrocodeine phosphate, dl-methyl-ephedrine hydrochloride and phenylpropanolamine hydrochloride.

Additionally, drugs that are suitable for the invention may be acidic, basic or amphoteric. Acidic drugs that can be used in the present invention include, for example dehydrocholic acid, diflunisal, ethacrynic acid, fenoprofen, furosemide, gemfibrozil, ibuprofen, naproxen, phenyloin, probenecid, sulindac, theophylline, salicylic acid and acetylsalicylic acid. Basic drugs that can be used in the present invention include, for example, acetophenazine, amitriptyline, amphetamine, benztropine, biperiden, bromodiphenhydramine, brompheniramine, carbinoxamine, chloperastine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, dicyclomine, diphemanil, diphenhydramine, doxepin, doxylamine, ergotamine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methylephedrine, methdilazine, methscopolamine, methysergide, metoprolol, nortriptylene, noscapine, nylindrin, orphenadrine, papaverine, pentazocine, phendimetrazine, phentermine, phenylpropanolamine, pyrilamine, tripelennamine, triprolidine, promazine, propoxyphene, propranolol, pseudoephedrine, pyrilamine, quinidine, scopolamine, dextromethorphan, morphine, oxycodone, levorphanol, chlorpheniramine, and codeine. Amphoteric drugs that can be used in the present invention include, for example, aminocaproic acid, aminosalicylic acid, isoxsuprine, melphalan, nalidixic acid, and paraminosalicylic acid.

Other drugs which may be used in the invention include, methylphenidate, dexmethylphenidate, oxymorphone, codeine, hydrocodone, chloropheniramine, niacin, aspirin, pharmaceutically acceptable salts thereof, and combinations thereof. Examples of such salts include, but are not limited to, methylphenidate HCl, dexmethylphenidate HCl, oxymorphone HCl, codeine phosphate, hydrocodone bitartrate, chlorpheniramine maleate, and salicylates. In an embodiment, a preferred drug is hydrocodone bitartrate.

The drug-ion exchange resin complex can be prepared by any suitable method. Typically, four general reactions are used for a basic drug: (a) resin (M⁺ form) plus drug (salt form); (b) resin (M⁺ form) plus drug (as free base); (c) resin (H⁺ form) plus drug (salt form); and (d) resin (H⁺ form) plus drug (as free base); wherein M⁺ is a cation, e.g., Na⁺. All of these reactions except (d) produce cationic by-products, and these by-products, by competing with the cationic drug for binding sites on the resin, reduce the amount of drug bound to the resin at equilibrium. For basic drugs, stoichiometric binding of drug to resin is accomplished only or preferably through reaction (d).

Four analogous binding reactions can be carried out for binding an acidic drug to an anion exchange resin. These are: (a) resin (X⁻ form) plus drug (salt form); (b) resin (X⁻ form) plus drug (as free acid); (c) resin (OH form) plus drug (salt form); and (d) resin (OH form) plus drug (as free acid); wherein X⁻ is an anion, e.g., Cl⁻. All of these reactions except (d) produce ionic (anionic) by-products, and the anions generated compete with the anionic drug for binding sites on the resin with the result that reduced levels of drug are bound at equilibrium. For acidic drugs, stoichiometric binding of drug to resin is accomplished only or preferably through reaction (d).

The binding of the drug to the ion exchange resin particles may be performed by any suitable process, for example, as a batch or column process. For example, a packed column of an ion exchange resin may be prepared and a solution of the drug passed through the column. Alternatively, a solution of the drug can be mixed with the ion exchange resin particles. The drug-ion exchange resin complex formed is collected by filtration and washed, e.g., with a suitable washing agent, such as ethanol, or preferably water, to ensure removal of any unbound drug. The complexes are dried, e.g., air-dried in trays at room temperature, if desired.

Any suitable amount of the drug can be loaded onto the ion exchange resin, e.g., from 1% to 90% by weight, typically from about 1% to about 50% by weight, preferably from about 5% to about 45% by weight, and more preferably, in embodiments, from about 10% to about 40% by weight of the drug-ion exchange resin complex particles.

It is believed that the acid form or the salt form of the ion exchange resin may have an influence on one or more properties of the sustained release drug particles. For example, in embodiments, the acid form tends to promote a slower release profile. This can be advantageously employed to prepare compositions capable of long-term sustained release and/or to administer smaller doses. Further, in embodiments, the salt form of the ion exchange resin tends to promote the long-term stability of the drug, especially an acid sensitive drug.

The drug-ion exchange resin complex particles may be optionally impregnated with a solvating agent. The impregnation of the solvating agent may be carried out by any suitable method. For example, the solvating agent can be added as an ingredient in the step where the drug is complexed with the ion exchange resin, or preferably, the particles can be treated with the solvating agent after completing the complexing reaction.

It is believed that the solvating agent, in certain embodiments, helps the particles retain their geometry, and enables the effective application of diffusion barrier coatings to such particles and prolong the release of the drug. Any suitable solvating agent, e.g., which can be a solid or liquid at ambient temperature, can be employed. Preferably, the solvating agent is a hydrophilic material, which can be a small molecule, a large molecule, or a polymer. An example of a solvating agent is a polyol or polyhydroxylic compounds. Examples of solvating agents include polyethylene glycol (e.g., one which is normally solid at ambient temperature), propylene glycol, mannitol, lactose, methylcellulose, hydroxypropylmethylcellulose, sorbitol, polyvinylpyrrolidone, carboxypolymethylene, xanthan gum, propylene glycol alginate, and combinations of these agents. The solvating agent may be present in the drug-ion exchange resin complex in any suitable amount, for example, in an amount of up to 100% by weight or more of the resin, typically from about 1% to about 100% by weight, and preferably from about 5% to about 30% by weight, and more preferably from about 5% to about 20% by weight of the ion exchange resin.

The drug-ion exchange resin complex particles (with or without solvating agent) are coated with a diffusion barrier comprising a water-permeable film-forming polymer. The film should be water-permeable. The polymer is insoluble, or has only limited solubility, in water and gastrointestinal fluids. The term “water-permeable” indicates that the fluids of the alimentary canal permeate or penetrate the film with or without dissolving the film or parts thereof. The polymer may be natural, synthetic, or semisynthetic. The polymer chosen as a film-forming polymer should have a sufficiently high molecular weight to facilitate film formation. Examples of suitable polymers include cellulose-based polymers such as ethylcellulose, methylcellulose, hydroxypropylmethylcellulose (HPMC), hydroxyethylcellulose (HEC), cellulose acetate phthalate, HEC phthalate, HPMC phthalate, or mixtures thereof. In an embodiment, the film-forming polymer can be an acrylic or methacrylic acid ester. Preferably the polymer for forming the diffusion barrier is ethyl cellulose, for example, an ethylcellulose having an ethoxyl group content of 44% to 47.5%, preferably from 45% to 46.5% by weight of the polymer.

In embodiments of the present invention, the diffusion barrier may include a plasticizer to improve one or more if its properties, e.g., reduce the glass transition temperature of the polymer, increase the flexibility of the film, and/or the adhesion of the film to the complex. The plasticizer may be included in the film in any suitable manner, for example, the polymer film may be exposed to a plasticizer (such as, e.g., a spray of a plasticizer solution) or preferably, the plasticizer may be added to the coating composition containing the film-forming polymer. The plasticizer can be present in the diffusion barrier in any suitable amount, for example, from about 5% to about 50%, preferably from about 10% to about 40%, and more preferably from about 15% to about 30% by weight of the film-forming polymer.

Any suitable pharmaceutically acceptable plasticizer can be used. Plasticizers may be chosen based on a number of parameters as appropriate. For example, plasticizers may be chosen based on their solubility parameter relative to that of the film-forming polymer. Examples of suitable plasticizers include water-insoluble plasticizers include high boiling alkyl or aryl esters, such as dibutyl sebacate, diethyl phthalate, triethyl citrate, tributyl citrate and triacetin, although it is possible that other water-insoluble plasticizers (such as acetylated monoglycerides, phthalate esters, castor oil or other vegetable oils, and the like) may be used. In a preferred embodiment, one or more of the above plasticizers, e.g., dibutyl sebacate or triethyl citrate, may be used in combination with ethylcellulose.

Any suitable coating procedure, preferably one that provides a contiguous and/or uniform coating on each particle of drug-ion exchange resin complex without significant agglomeration of particles, may be used. For example, coatings may be applied with a fluid-bed coating apparatus having a “Wurster configuration”. Measurement of particle size and size distribution may be carried out before and after coating to show that agglomeration of particles is insignificant.

In accordance with a preferred embodiment of the invention, the film-forming polymer is applied as an aqueous dispersion. The aqueous dispersion of the film-forming polymer can be prepared by any suitable method, for example, (i) by dissolving the polymer in a minimum amount of organic solvent, and emulsifying the resulting solution with water in the presence of a suitable surfactant and a stabilizer; (ii) by homogenizing a mixture of a hot melt of the polymer, a plasticizer, and any other additives (such as stabilizers or surfactants), and diluting the resulting mixture with water or an alkaline aqueous solution; or (iii) any variations or combinations of (i) and (ii).

Alternatively, a commercially available aqueous dispersion may be used. Examples of suitable aqueous dispersions of ethylcellulose include AQUACOAT™ (FMC Corp., Philadelphia, Pa.) and SURELEASE™ (Colorcon, Inc., West Point, Pa.). Prior to using AQUACOAT dispersion, it is usually mixed with a suitable plasticizer.

The coating composition may contain other additives, e.g., pigments or other substances, for example, appearance or taste, to alter or improve one or more characteristics of the coating or the drug formulation.

The coating composition may be applied at any desired coat weight. Optimum coat weight and coat thickness may be determined for each drug-ion exchange resin complex and will generally depend on a number of factors including, for example, the desired drug release characteristics. For example, the coat weight can be from about 5% to about to 50% of the complex.

In an embodiment, the present invention provides a method for manufacturing coated particles for use in the manufacture of a prolonged or sustained release preparation comprising contacting particles of an ion exchange resin with a pharmaceutically active drug to form a drug-ion exchange resin complex wherein the particle size of the ion exchange resin particles is from about 30 microns to about 500 microns; and coating the drug-ion exchange resin complex with an aqueous suspension of a water-permeable film-forming polymer which contains no substantial traces of an organic solvent such that the resulting coating has an average thickness of at least about 10 microns.

The drug-ion exchange resin complex particles may be prepared by the use of any suitable technique and equipment. For example, the drug-ion exchange resin complex is prepared by suspending the resin particles in a suitable amount of purified water followed by addition of the drug with stirring. After thorough mixing, the solids are allowed to settle, the supernatant liquid is decanted, and the resulting complex is washed, e.g., with purified water. If a solvating agent is to be included in the complex, the solvating agent or a solution of the solvating agent is added to the complex, mixed thoroughly, and the mixture dried if desired. Alternatively, the complex can be suspended in water and the solvating agent is added to the suspension. After thorough mixing, the suspension is filtered to obtain a solvating agent-treated (or impregnated) drug-ion exchange resin complex.

The complex, which may be impregnated or non-impregnated with a solvating agent, is screened to remove any lumped material or particles of undesired size. The screened product is coated with an aqueous dispersion of diffusion barrier coating polymer material, optionally containing other additives such as plasticizers. The coating may be applied from a bottom spray or top spray configuration. If necessary, the coated drug-ion exchange resin complex may be screened to any desired size.

Optionally, after coating the drug-ion exchange resin complex, the coated complex may be cured at a suitable temperature and for a suitable period of time. Curing is intended to heat the recently (nascently or incipiently) formed diffusion barrier such that one or more properties of the barrier are improved or stabilized. For example, it is contemplated that the polymer may achieve a low energy configuration. The polymer film may adhere to, or lay flat on, the complex, or the porosity of the barrier may change. The product may be cured by any suitable method, e.g., in a fluid bed, at a suitable temperature, e.g., from about 35° C. to about 75° C.

The present invention further provides a pharmaceutical composition comprising ion exchange resin particles having particle sizes from about 30 microns to about 500 microns; at least one pharmaceutically active drug releasably bound to the ion exchange resin particles to form a drug-ion exchange resin complex; and a pharmaceutically acceptable carrier, wherein the drug-ion exchange resin complex is coated with an aqueous based diffusion barrier which comprises from about 1% to about 60%, by weight of the ion exchange resin particles, of a water-permeable film-forming polymer. In an embodiment, the pharmaceutically acceptable carrier is a liquid.

The coated drug-ion exchange resin particles according to the present invention are suitable for preparing solid or liquid formulations, e.g., oral formulations such as tablets, capsules, granules, lozenges, and bulk powders, and liquid forms such as syrups and suspensions. In an embodiment of the invention, the coated drug-ion exchange complex particles are suspended in an essentially aqueous vehicle to provide an oral suspension. Liquid forms such as syrups and suspensions can be prepared to contain any suitable amount of the coated drug-ion exchange resin complex.

Liquid oral dosage forms include aqueous and nonaqueous solutions, emulsions, suspensions, and solutions and/or suspensions reconstituted from non-effervescent granules, containing suitable solvents preservatives, emulsifying agents, suspending agents, diluents, sweeteners, coloring agents, and flavoring agents. In a preferred embodiment, the formulation contains none or very low levels of ionic ingredients and/or water-miscible organic solvents. For example, if an organic solvent such as alcohol is present, it should be limited to those levels that do not cause dissolution or degradation of the diffusion barrier.

In preparing the liquid oral dosage forms, the coated drug-ion exchange resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. Preferably, the carrier is a suspension of the pharmaceutical composition in an aqueous vehicle containing a suitable suspending agent. Suitable suspending agents include AVICEL™ RC-591 (a microcrystalline cellulose/sodium carboxymethylcellulose mixture available from FMC), guar gum, starch, xanthan gum, and the like. The amount of water in the compositions can vary over quite a wide range depending upon the total weight and volume of the drug-ion exchange resin complex and other optional inactive ingredients.

Although water itself may make up the entire carrier, typical liquid formulations contain a co-solvent, for example, propylene glycol, glycerin, sorbitol solution and the like, to assist solubilization and incorporation of water-insoluble ingredients, such as flavoring oils and the like into the composition. In general, therefore, the compositions of this invention contain from about 0.1% about 20%, preferably from about 0.5% to about 15%, and more preferably from about 1% to about 10%, v/v, of the co-solvent.

The term “no substantial traces of organic solvent” means that the film-forming polymer has none or low level of organic solvents, particularly volatile organic solvents, for example, less than about 1% by weight of organic solvents. Typical organic solvents include, but are not limited to, volatile organic solvents such as chloroform, methylene chloride, acetone, tetrahydrofuran, and the like.

The compositions of this invention may optionally contain one or more other known therapeutic agents, for example, a coated hydrocodone bitartrate ion exchange complex in combination with another complexed drug, e.g., chlorpheniramine complexed with an ion exchange resin. Other optional ingredients known to the pharmacist's art may also be included in amounts generally known for these ingredients, for example, natural or artificial sweeteners, flavoring agents, colorants and the like to provide a palatable and pleasant looking final product, antioxidants, for example, butylated hydroxy anisole or butylated hydroxy toluene, and preservatives, for example, methyl or propyl paraben or sodium benzoate, to prolong and enhance shelf life.

The present invention further provides a method for the controlled or sustained administration of a drug comprising administering to a patient a therapeutically acceptable dose of a composition comprising a diffusion barrier coated drug-ion exchange resin complex wherein the diffusion barrier is present in an amount of about 1% to about 60% by weight of the drug-ion exchange resin complex and the diffusion barrier comprises a water-permeable film-forming polymer and contains no substantial traces of an organic solvent.

The composition of the invention provides controlled release of active drug in vivo and in vitro. For example, in an embodiment, the drug is released in vivo or in vitro over a period of about 4 hours; in another embodiment, the drug is released in vivo or in vitro over a period of about 12 hours; and in yet another embodiment, the drug is released in vivo or in vitro over a period of 24 hours. Drug-ion exchange resin complexes release the drug in the patient, such as, for example, in the gastrointestinal tract. The diffusion barrier does not rupture when exposed to water or gastrointestinal fluids.

The coated drug-ion exchange resin complex may also be provided with one or more other coatings, e.g., an enteric coating.

The following examples further illustrate the invention but, of course, should not be construed as in any way limiting its scope.

### EXAMPLE 1

This example illustrates the preparation of a coated drug-ion exchange resin complex in accordance with an embodiment of the invention. A coated drug-ion exchange resin complex was prepared by employing hydrocodone bitartrate as the drug, DOWEX 50WX8H as the ion exchange resin, polyethylene glycol as the solvating agent, and ethylcellulose (AQUACOAT suspension containing dibutyl sebacate plasticizer) as the film-forming polymer.

DOWEX 50WX8H resin (100-200 mesh; 50% water content; 2 kg) was suspended in 1 L of water. Hydrocodone bitartrate (HB; about 106 g) was added to the suspension over 15 minutes and stirred for 30 minutes, and the mixture was allowed to settle. The supernatant liquid was drained off and the resulting cake was suspended in 500 mL water. About 106 g of HB was added over 15 minutes and stirred for 30 minutes. The suspension was filtered in a Buchner funnel, washed with water and dried in a fluid bed to obtain 1339.4 g of the complexed product. This product was suspended in 1339.4 g water and mixed with 334.9 g of polyethylene glycol (PEG 3350). The suspension was filtered in a Buchner funnel and the wet cake was dried in a fluid bed to obtain 1406.9 g of a dry product (Hydrocodone Polistirex or HP) (Lot 3570-145A) with an estimated hydrocodone bitartrate content of 18%.

A laboratory-scale fluid bed processor (GPCG-1 made by Glatt Air Techniques, Inc.) was assembled to perform the ethylcellulose coating with the bottom-spray configuration (“Wurster configuration”). The aqueous ethylcellulose suspension (AQUACOAT; 667.1 g) was mixed with 92.6 g of deionized water and 40.5 g dibutyl sebacate for about 1 hour and allowed to stand overnight. The dry HP (Lot 3570-145A; 559 g) was fluidized in the fluid bed processor and the ethylcellulose suspension was applied to achieve a nominal weight gain of about 43%. When all the suspension had been applied, the resulting product was cured in the fluid bed at about 55° C. for about 82 minutes, and cooled to room temperature to obtain 725.2 g of the final product (Lot 3570-165). Some of the key coating parameters were:

| | |
|---|---|
| Inlet air temperature: | About 50° C. |
| Product bed temperature: | About 43° C. |
| Exhaust air temperature: | 37-45° C. |
| Spray rate: | 3.7 g/minute |
| Air flow velocity: | 5-8.0 m/sec |
| Atomizing air pressure: | 1.5 bars |
| Duration of coating run: | About 212 minutes. |

The release profile of hydrocodone was evaluated using a VanKel dissolution tester set up with the following test parameters:
Medium: 500 mL 0.1N HCl
No. of vessels: 2
Speed: 50 rpm
Apparatus: USP Type 2 (paddles)
Sampling Time: 15, 30, 45, 60, 90, 120, and 180 minutes.

The samples were analyzed by UV spectroscopy to determine the release profile. Sustained release was demonstrated as shown by the data set forth in Table 1.

TABLE 1
Hydrocodone release profile of coated drug-ion exchange resin complex.

| Ves- | Approximate % of hydrocodone released | | | | | | |
|---|---|---|---|---|---|---|---|
| sel | 15 min | 30 min | 45 min | 60 min | 90 min | 120 min | 180 min |
| 1 | 6.7 | 8.7 | 11.3 | 11.3 | 14.7 | 16.7 | 20.7 |
| 2 | 6.7 | 10.0 | 11.3 | 13.3 | 15.3 | 17.3 | 21.3 |

### EXAMPLE 2

This example illustrates the preparation of a coated drug-ion exchange resin complex in accordance with another embodiment of the invention. A coated drug-ion exchange resin complex was prepared by employing hydrocodone bitartrate as the drug, AMBERLITE IRP-69 as the ion exchange resin (as the sodium salt), polyethylene glycol as the solvating agent, and ethylcellulose (SURELEASE) as the film-forming polymer.

Hydrocodone bitartrate (203 g) was dissolved in 2 L of deionized water. The AMBERLITE IRP-69 resin (particle size range of US Standard Mesh 100-400) was sieved to obtain dry particles in the size range of US Standard Mesh 100-200. The sieved resin (1 kg) was suspended in 2 L of water and mixed for 1 hour with the hydrocodone bitartrate solution made above. The resulting complex was filtered in a sintered glass funnel and washed 3 times with 1 L water each. The resulting wet cake was suspended in 600 mL water containing 272 g of polyethylene glycol (PEG 4000) and mixed by hand for 10 minutes, allowed to stand for 30 minutes, and mixed again by hand for 10 minutes. The resulting wet cake was filtered and dried in a fluid-bed to obtain 1.22 kg of the PEG-impregnated complex (Lot 3880-184). The hydrocodone content of the complex was 12.3%.

A laboratory-scale fluid bed processor (described as in Example 1) was assembled to perform coating. The SURELEASE aqueous ethylcellulose suspension (600 g) was mixed with 400 g of deionized water. The dry complex (Lot 3880-184; 600 g) was fluidized in the fluid bed processor and 800 g of the SURELEASE suspension prepared above was applied to achieve a nominal weight gain of about 20%. When all the suspension had been applied, the product was cooled in the fluid bed to room temperature to obtain 648.7 g of the final product (Lot 3900-67B). HPLC assay showed the hydrocodone bitartrate content to be 10.6%. Some of the key coating parameters were:

| | |
|---|---|
| Inlet air temperature: | About 52° C. |
| Product bed temperature: | About 38-41° C. |
| Exhaust air temperature: | 35-38° C. |
| Spray rate: | 3.1 g/minute |
| Air flow velocity: | 2-2.5 m/sec |
| Atomizing air pressure: | 1.5 bars. |

The release profile of hydrocodone from the coated complex was evaluated using a VanKel dissolution tester set up with the test parameters given below. The samples were analyzed by HPLC to determine the release profile. Sustained release was demonstrated as shown by the data set forth in Table 2.
Medium: 500 mL 0.1N HCl
No. of vessels: 3
Speed: 100 rpm
Apparatus: USP Type 2 (paddles)
Sampling Time: 15, 30, 60, 120, and 180 minutes.

### EXAMPLE 3

This example illustrates the preparation of a coated drug-ion exchange resin complex in accordance with yet another embodiment of the invention. A coated drug-ion exchange resin complex was prepared by employing hydrocodone bitartrate as the drug, DOWEX 50WX8H as the ion exchange resin in acid form, polyethylene glycol as the solvating agent, and ethylcellulose (SURELEASE) as the film-forming polymer. A coated complex having a nominal weight gain of 35% was prepared.

The DOWEX resin (50% water content; US Standard Mesh #100-200; 3000 g) was suspended in 2 L of water and mixed with 300 g of hydrocodone bitartrate dissolved in 3 L of water. The mixture was stirred for 2 hours and the resulting complex was filtered in a sintered glass funnel. After washing with 5 L water, the wet cake was dried in a fluid bed to yield 1.78 kg of the complex Hydrocodone Polistirex (HP; Lot 4687-48). The HP (Lot 4687-48; 1.78 kg) was mixed in a KitchenAid mixer with 750 mL water containing 336 g of PEG 4000 over 15 minutes and allowed to stand for about 20 minutes, followed by hand-mixing for 5 minutes. The product was isolated as a wet cake and dried in a fluid-bed to give 1.86 kg of product. The dry product was sieved through a #60 mesh and #200 mesh screens to yield 1.92 kg of HP-PEG complex (Lot 4687-49). The hydrocodone content of Lot 4687-49 was 6.28%.

The HP-PEG complex (Lot 4687-49; 600 g) was coated in a laboratory-scale fluid bed processor as described in Example 2. The aqueous ethylcellulose suspension (840 g) was mixed with 560 g of deionized water. The total amount of the SURELEASE suspension applied was 1400 g to achieve a nominal weight gain of about 35%. When the required suspension had been applied, the product was cooled in the fluid bed to room temperature to obtain 761.5 g of a dry powder. The powder was sieved through a US Standard #60 screen to give 655.6 g of HP-PEG complex (Lot 3900-151). HPLC assay showed the hydrocodone bitartrate content of Lot 3900-151 to be 5.56%. Some of the key coating parameters were:

| | |
|---|---|
| Inlet air temperature: | About 51-56° C. |
| Product bed temperature: | About 38-41° C. |
| Exhaust air temperature: | About 39-41° C. |
| Spray rate: | About 3.3 g/minute |
| Air flow velocity: | 2-3.3 m/sec |
| Atomizing air pressure: | 1.5 bars. |

A small sample from coated particles from Lot 3900-152 was sieved through US Standard Mesh #100 and #200 screens. The −100/+200 fraction was used to perform a dissolution test using a VanKel dissolution tester set up with the test parameters as set forth below. The dissolution samples were analyzed by HPLC to determine the release profile. Sustained release was demonstrated as shown by the data set forth in Table 2.
Medium: 500 mL 0.1N HCl
No. of vessels: 3
Speed: 150 rpm
Apparatus: USP Type 2 (paddles)
Sampling Time: 15, 30, 60, 120, and 180 minutes.

### EXAMPLE 4

This example illustrates the preparation of a coated drug-ion exchange resin complex in accordance with still another embodiment of the invention. A coated drug-ion exchange resin complex was prepared by employing hydrocodone bitartrate as the drug, DOWEX 50WX8H as the ion exchange resin in acid form, polyethylene glycol as the solvating agent, and ethylcellulose (AQUACOAT) the film-forming polymer. A coated complex having a nominal weight gain of 35% was prepared.

The HP-PEG complex (Lot 4687-49; 600 g) as prepared in Example 3 was coated in a laboratory-scale fluid bed processor as described in Example 3. The aqueous ethylcellulose suspension (600 g) was mixed with 45 g of dibutyl sebacate overnight and diluted with 105 g of water. 700 g of the above suspension was applied to 600 g of the HP-PEG complex to achieve a nominal weight gain of about 35%. The product was cooled in the fluid bed to room temperature to obtain 736.6 g of a dry powder (Lot 3900-155). HPLC assay showed a hydrocodone bitartrate content of Lot 3900-155 to be 6.48%. Some of the key coating parameters were:

| | |
|---|---|
| Inlet air temperature: | About 45-47° C. |
| Product bed temperature: | About 39-41° C. |
| Exhaust air temperature: | About 39-40° C. |
| Spray rate: | About 3.3 g/minute |
| Air flow velocity: | 3.3-4.0 m/sec |
| Atomizing air pressure: | 1.5 bars. |

A small sample from coated particles from Lot 3900-155 was sieved through US Standard Mesh #100 and #200 screens. The −100/+200 fraction was used to perform a dissolution test using a VanKel dissolution tester set up with the test parameters as in Example 3. The dissolution samples were analyzed by HPLC to determine the release profile. Sustained release was demonstrated as shown by the data set forth in Table 2.

### EXAMPLE 5

This example illustrates the preparation of a coated drug-ion exchange resin complex in accordance with a further embodiment of the invention. A coated drug-ion exchange resin complex was prepared by employing hydrocodone bitartrate as the drug, AMBERLITE IRP-69 as the ion exchange resin (as the acid form), polyethylene glycol as the solvating agent, and ethylcellulose (SURELEASE) as the film-forming polymer. A coated complex having a nominal weight gain of 35% was prepared.

The AMBERLITE IRP-69 resin (sodium salt) was sieved to give particles in the size range of US Standard Mesh 100-200. The sieved resin (1 kg) was suspended in 3 L of water and the pH adjusted to 2.29 by mixing with 10N sulfuric acid. The suspension was filtered through a coarse sintered glass funnel and washed 3 times with 1500 mL water each. The resulting wet cake was suspended in 3 L water and mixed with 17 mL of ION sulfuric acid to reach a pH of 2.35 in the resin suspension. Hydrocodone bitartrate (HB; 200 g) was dissolved in 3 L of water and the solution was added to the resin suspension, and mixed for 2 hours. The resulting complex was filtered in a sintered glass funnel and washed 3 times with 1.5 L water each. The resulting wet cake was dried in a fluid bed to yield 1.15 kg of the Hydrocodone Polistirex (HP; Lot 4687-60). The HP (1.09 kg) was mixed in a KitchenAid mixer with 600 mL water containing 272 g of PEG 4000 and allowed to stand for 15 minutes, followed by hand mixing for 2 minutes. This was followed by mixing in a KitchenAid mixer for 5 minutes. The product was isolated as a wet cake, sieved through a #4 mesh screen and dried in fluid-bed. The dry product was sieved through a #40 mesh screen using an Erweka oscillator and then through a 200 mesh screen to give 1.21 kg of HP-PEG complex (Lot 4687-62). The hydrocodone bitartrate content of Lot 4687-62 was 11.65%.

The HP-PEG complex obtained above (600 g) was coated in a laboratory-scale fluid bed processor as described in Example 2. The aqueous ethylcellulose suspension (840 g) was mixed with 560 g of deionized water, and the resulting suspension (1400 g) was applied to achieve a nominal weight gain of about 35%. The resulting product was cooled in the fluid bed to room temperature to obtain 767.6 g of the final product (Lot 3900-178C). HPLC assay showed a hydrocodone bitartrate content of Lot 3900-178C to be 8.83%. Some of the key coating parameters were:

| | |
|---|---|
| Inlet air temperature: | About 54-56° C. |
| Product bed temperature: | About 37-41° C. |
| Exhaust air temperature: | 37-38° C. |
| Spray rate: | 1.9-3.4 g/minute |
| Air flow/velocity: | 2-2.5 m/sec |
| Atomizing air pressure: | 1.5 bars. |

The release profile of hydrocodone was evaluated using a VanKel dissolution tester set up with the test parameters as in Example 3. The samples were analyzed by HPLC to determine the release profile. Sustained release was demonstrated as shown by the data in Table 2.

TABLE 2
Hydrocodone release profile from coated drug-ion
exchange resin complex.

| | Percent of hydrocodone released (std. dev.) | | | |
|---|---|---|---|---|
| | Example 2 | Example 3 | Example 4 | Example 5 |
| Sampling | Lot | Lot | Lot | Lot |
| Time | 3900-67B | 3900-151 | 3900-155 | 3900-178C |
| 15 minutes | 30.1 (6.9) | 2.4 (0.6) | 47.0 (1.6) | 18.1 (0.7) |
| 30 minutes | 45.6 (10.0) | 4.6 (0.6) | 65.0 (1.1) | 29.4 (1.9) |
| 60 minutes | 58.6 (11.8) | 7.5 (1.1) | 76.5 (0.4) | 43.2 (1.5) |
| 120 minutes | 69.4 (12.2) | 10.8 (1.3) | 79.6 (0.03) | 56.4 (1.5) |
| 180 minutes | 73.1 (11.8) | 12.7 (1.5) | 80.8 (0.4) | 63.8 (1.4) |

The foregoing shows that the embodiments illustrated above released the drug in a sustained manner.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An oral pharmaceutical composition comprising ion exchange resin particles having particle sizes from about 30 microns to about 500 microns; at least one pharmaceutically active drug releasably bound to the particles to form a drug-ion exchange resin complex, wherein the drug-ion exchange resin complex is coated with an aqueous based diffusion barrier which comprises from about 1% to about 60%, by weight of the resin particles, of a water-permeable film-forming polymer which contains no substantial traces of an organic solvent.

2. The composition of claim 1, wherein the particle size of the ion exchange resin particles is from about 40 microns to about 200 microns.

3. The composition of to claim 1, wherein the ion exchange resin particles are regularly shaped, irregularly shaped, or both.

4. The composition of claim 1, wherein the ion exchange resin has an ion-exchange capacity of less than 6.0 meq/gram.

5. The composition of claim 1, wherein the drug comprises from about 1% to about 50% by weight of the drug-ion exchange resin complex.

6. The composition of claim 1, wherein the water-permeable film-forming polymer comprises ethylcellulose.

7. The composition of claim 1, which provides a controlled release of active drug in vivo.

8. The composition of claim 1, wherein the drug-ion exchange resin complex contains a solvating agent.

9. The composition of claim 8, wherein the solvating agent comprises polyethylene glycol.

10. The composition of claim 1, wherein the pharmaceutically active drug is selected from the group consisting of antitussive expectorants, bronchodilators, antihistamines, digestive tract antispasmodics, antipsychotic drugs, antianxiety drugs, antidepressants, antipyretic analgesics, opioid analgesic drugs, coronary dilators, hypotensive drugs, peripheral vasodilators/vasoconstrictors, antibiotics, chemo-therapeutic drugs, antituberculosis drugs, and antiprotozoan drugs.

11. The composition of claim 1, wherein the pharmaceutically active drug is selected from the group consisting of dehydrocholic acid, diflunisal, ethacrynic acid, fenoprofen, furosemide, gemfibrozil, ibuprofen, naproxen, phenyloin, probenecid, sulindac, theophylline, salicylic acid, acetylsalicylic acid, acetophenazine, amitriptyline, amphetamine, benztropine, biperiden, bromodiphenhydramine, brompheniramine, carbinoxamine, chlorcyclizine, chlorpheniramine, chlorphenoxamine, chlorpromazine, clemastine, clomiphene, clonidine, codeine, cyclizine, cyclobenzaprine, cyproheptadine, desipramine, dexbrompheniramine, dexchlorpheniramine, dextroamphetamine, dextromethorphan, diazepam, dicyclomine, diphemanil, diphenhydramine, doxepin, doxylamine, ergotamine, fexofenadine, fluphenazine, haloperidol, hydrocodone, hydroxychloroquine, hydroxyzine, hyoscyamine, imipramine, levopropoxyphene, maprotiline, meclizine, mepenzolate, meperidine, mephentermine, mesoridazine, methadone, methdilazine, methscopolamine, methysergide, metoprolol, nortriptylene, noscapine, nylindrin, orphenadrine, papaverine, pentazocine, phendimetrazine, phentermine, phenylpropanolamine, pyrilamine, tripelennamine, triprolidine, promazine, propoxyphene, propanolol, pseudoephedrine, pyrilamine, quinidine, scopolamine, dextromethorphan, chlorpheniramine, aminocaproic acid, aminosalicylic acid, hydromorphone, isoxsuprine, levorphanol, melphalan, morphine, nalidixic acid, oxycodone, tramadol, and paraminosalicylic acid, and any combination thereof, and pharmaceutically acceptable salts thereof.

12. The composition of claim 1, which is a liquid.

13. A method for manufacturing sustained release drug particles for use in the manufacture of a sustained release preparation comprising contacting particles of an ion exchange resin with a pharmaceutically active drug to form a drug-ion exchange resin complex wherein the particle size of the ion exchange resin particles is from about 30 microns to about 500 microns; and coating the drug-ion exchange resin complex with an aqueous suspension of a water-permeable film-forming polymer which contains no substantial traces of an organic solvent such that the resulting coating has an average thickness of at least about 10 microns.

14. The method of claim 13, wherein the particle size of the ion exchange resin particles is from about 40 microns to about 200 microns.

15. The method of claim 13, wherein the drug comprises from about 1% to about 50% by weight of the drug-ion exchange resin complex.

16. The method of claim 13, wherein the water-permeable film-forming polymer comprises ethylcellulose.

17. The method of claim 13, comprising impregnating a solvating agent on to the drug-ion exchange resin complex.

18. The method of claim 17, wherein the solvating agent is polyethylene glycol.

19. A pharmaceutical composition comprising: ion exchange resin particles having particle sizes from about 30 microns to about 500 microns; at least one pharmaceutically active drug releasably bound to the ion exchange resin particles to form a drug-ion exchange resin complex; and a pharmaceutically acceptable carrier, wherein the drug-ion exchange resin complex is coated with an aqueous based diffusion barrier which comprises a water-permeable film-forming polymer in an amount of from about 1% to about 60%, by weight of the ion exchange resin particles.

20. The composition of claim 19, wherein the pharmaceutically acceptable carrier is a liquid.

21. The pharmaceutical composition of claim 19, wherein the composition is a liquid.

22. A method for the controlled administration of a drug to a patient comprising administering to the patient a therapeutically acceptable dose of a composition comprising a diffusion barrier coated drug-ion exchange resin complex wherein the diffusion barrier is present in an amount of about 1% to about 60% by weight of the drug-ion exchange resin complex and the diffusion barrier comprises a water-permeable film-forming polymer and contains no substantial traces of an organic solvent.

23. The method of claim 22, wherein the water-permeable film-forming polymer is ethylcellulose.

24. The method of claim 22, wherein the diffusion barrier coated drug-ion exchange resin complex includes a solvating agent.

25. The method of claim 24, wherein the solvating agent is polyethylene glycol.

26. The method of claim 22, wherein the drug is released in vivo over a period of about 4 hours.

27. The method of claim 22, wherein the drug is released in vivo over a period of about 12 hours.

28. The method of claim 22, wherein the drug is released in vivo over a period of 24 hours.

29. The method of claim 22, wherein the ion exchange resin has a particle size from about 30 microns to about 500 microns.

30. The method of claim 22, wherein the ion exchange resin has a particle size from about 40 microns to about 150 microns.

31. The method of claim 22, wherein the composition is a liquid.

32. A drug particle suitable for forming a sustained release oral pharmaceutical composition comprising a drug-ion exchange resin complex, a solvating agent, and a water-permeable diffusion barrier surrounding at least a portion of the drug-ion exchange resin complex;
wherein the diffusion barrier comprises a film-forming polymer and is free or substantially free of traces of organic solvents.

33. A sustained release oral pharmaceutical composition comprising the drug particle of claim 32.

34. An oral pharmaceutical composition comprising ion exchange resin particles having particle sizes from 30 microns to about 500 microns; at least one pharmaceutically active drug releasably bound to the particles to form a drug-ion exchange resin complex, wherein the drug-ion exchange resin complex is coated with an aqueous based diffusion barrier which comprises from about 1% to about 60%, by weight of the resin particles, of a water-permeable film-forming polymer.
